(19) United States
(12) Patent Application Publication
(10) Pub. No.: US 2020/0172629 A1
(43) Pub. Date: Jun. 4, 2020

(54) T CELL ENGAGING AGENTS AND METHODS OF USE THEREOF

(71) Applicant: Washington University, St. Louis, MO (US)

(72) Inventors: Abdel Kareem Azab, St. Louis, MO (US); Kinan Alhallak, St. Louis, MO (US)

(21) Appl. No.: 16/698,285

(22) Filed: Nov. 27, 2019

Related U.S. Application Data
(60) Provisional application No. 62/772,152, filed on Nov. 28, 2018.

(51) Int. Cl. C07K 16/28; C07K 16/46; C07K 16/30; A61K 47/68; A61K 47/69; A61P 35/02

(52) U.S. Cl. CPC C07K 16/2896; C07K 16/2878; C07K 16/2803; C07K 16/468; A61K 2039/505; A61K 47/6898; A61K 47/6929; A61P 35/02; C07K 16/30

(57) ABSTRACT

The present disclosure discloses compositions, and methods of making and using nanoparticles to treat cancer. Among the various aspects of the present disclosure is the provision of a nanoparticle composition and methods of using same. For example, the nanoparticle composition can comprise a nanoparticle and antibodies conjugated to the nanoparticle surface, an antibody can recognize an epitope on cancer cells (e.g., multiple myeloma), and another antibody can engage T cells.

### CROSS REFERENCE TO RELATED APPLICATIONS

This application claims the benefit of U.S. Provisional Application No. 62/772,152, filed Nov. 28, 2018, the disclosure of which is hereby incorporated by reference in its entirety.

### FIELD OF THE TECHNOLOGY

The present disclosure generally relates to targeted cancer treatment. In particular, the present disclosure provides a multi-specific nanoparticle system for the treatment of heterogeneous cancer cells.

### BACKGROUND

T cell-based immunotherapy such as chimeric antigen receptor (CAR)-T cells is an emerging strategy in the treatment of cancer. CAR-T cells are autologous T cells that have been virally transfected to express an engineered CAR construct, containing a synthesized fragment that targets the desired surface antigen on the cancer cell. Several studies have shown promising preclinical and clinical results with the use of this technology. The main disadvantages of this technology relative to traditional therapies include toxicity, the long-term safety profile of the viral vector, the need to perform quality control testing frequently throughout the production of CAR-T cells, the high costs associated with this technique due to the need of extensive labor and expensive facility equipment, and the inability to target multiple tumor antigens with one CAR-T cell.

In addition to CAR-T cells, T cell-based therapy can be pursued with bispecific T cell engagers (BITEs). BITE molecules are constructed from two different recognition domains connected by a protein linker. One of the domains recognizes a tumor-associated surface antigen, while the other domain recognizes a T cell surface marker, in order to activate and redirect cytotoxic T cells to tumor cells. BITES demonstrate high potency and efficacy against tumor cells and exploit the use of endogenous T cells, circumventing the limitation of genetically engineering extracted patient T cells to express CARs. The disadvantages of the BITE, however, include severe toxicity at high doses, expensive costs with regards to its labor and production, and the inability to target multiple cancer surface markers. One common disadvantage of the CAR-T and BITE technologies is the ability to only target a single epitope on the cancer cell, while it is evidently known that cancer cells express a landscape of heterotypic genes and moieties. This may cause these technologies to only affect some parts of the tumor and not others.

Overall, the care of patients with cancer is complex and in general focuses on treatment of the disease process and any associated complications. Despite the introduction of novel therapies, many cancer patients relapse, due to therapy-resistant stem-cell-like cancer cells and minimal residual disease (MRD). Minimal residual disease (MRD) is a term used to describe the small number of cancer cells in the body which remain after cancer treatment. Genetic and phenotypic heterogeneity of cancer cells is a major contributor to therapy resistance and disease relapse.

Therefore, a new strategy to specifically target heterogeneous cancer cells is needed.

### SUMMARY

The present disclosure is based on the unexpected discovery of a multi-specific nanoparticle system with enhanced ability redirect T cell populations to heterogeneous target cells. The nanoparticle system of the disclosure provides improved therapeutic agents in treatment of cancer patients.

Accordingly, one aspect of the present disclosure features a nanoparticle composition comprising: (a) at least one antigen binding moiety which specifically binds to an antigen on a cancer cell; and (b) a T cell moiety which specifically binds to a T cell; wherein the antigen binding moiety and the T cell moiety are conjugated to the surface of a nanoparticle. In some examples, the nanoparticle can have 2, 3, 4, or more antigen binding moieties, wherein a first antigen binding moiety binds to a first antigen on a target cell, a second antigen binding moiety binds to a second antigen on a target cell, a third antigen binding moiety binds to a third antigen on a target cell, etc. In one aspect, the first, second, and/or third cancer cells are heterogeneous cancer cells (e.g., have distinct molecular signatures) in the same subject.

In some embodiments, the compositions disclosed herein comprise an antigen binding moiety which is an antibody, a T cell moiety which is an antibody, or both the antigen binding moiety and the T cell moiety are antibodies. In some examples, the compositions comprise at least one antigen biding moiety selected from an antibody which specifically binds CD38, an antibody which specifically binds B Cell Maturation Antigen (BCMA), an antibody which specifically binds to CD20, an antibody which specifically bind to CD33, an antibody which specifically bind to EpCAM or an antibody which specifically bind to CS1. In some examples, the T cell moiety comprises an antibody which specifically binds CD3.

In some instances, the compositions disclosed herein comprise a liposomal nanoparticle. In some examples, the liposomal nanoparticle is avidin-conjugated. In some examples, the antigen binding moiety and T cell moiety are biotinylated.

In other embodiments, the compositions of the disclosure provide pharmaceutical compositions, comprising: the nanoparticle composition as disclosed herein and a pharmaceutically acceptable carrier.

In still other embodiments, one aspect of the present disclosure provides a method of killing a cancer cell in a subject. In some embodiments, the method disclosed herein may comprise: administering a therapeutically effective amount of a nanoparticle composition comprising, (a) at least one antigen binding moiety which specifically binds an antigen on a cancer cell, and (b) a T cell moiety which specifically binds to a T cell; wherein the antigen binding moiety and the T cell moiety are conjugated to the surface of a nanoparticle. In some examples, the composition redirects the T cell to the cancer cell and increases T cell mediated lysis of the cancer cell relative to a subject who has not been administered the nanoparticle composition.

In some examples, the methods include administering a nanoparticle with 2, 3, 4, or more antigen binding moieties, wherein a first antigen binding moiety which binds to a first antigen on a target cell, a second antigen binding moiety which binds to a second antigen on a target cell, a third antigen binding moiety which binds to a third antigen on a target cell, etc. In one aspect, the first, second, and/or third cancer cells are heterogeneous cancer cells (e.g., have distinct molecular signatures) in the same subject. Accordingly, the methods disclosed herein are useful for killing cancer cells with distinct molecular signatures (e.g., differentially expressed cell surface markers) in the same subject.

In some embodiments, the methods disclosed herein may comprise administering a nanoparticle composition comprising an antigen binding moiety which is an antibody, a T cell moiety which is an antibody, or both the antigen binding moiety and the T cell moiety are antibodies. In some examples, the antigen biding moiety comprise an antibody which specifically binds CD38, an antibody which specifically binds B Cell Maturation Antigen (BCMA), an antibody which specifically binds to CD20, an antibody which specifically bind to CD33, an antibody which specifically bind to EpCAM or an antibody which specifically bind to CS1. In some examples, the T cell moiety comprises an antibody which specifically binds CD3.

In some instances, the methods disclosed herein comprise administering a liposomal nanoparticle, where the liposomal nanoparticle is avidin-conjugated. In some examples the antigen binding moiety and T cell moiety are biotinylated. In some examples the subject has cancer and the nanoparticles redirects T cells to the cancer cells, wherein the cancer cells are, for example multiple myeloma, lymphoma, leukemia, or a solid tumor such as a lung cancer, cervical cancer, colon cancer, breast cancer or prostate cancer.

In one aspect of the disclosure, administration of the nanoparticle compositions as disclosed herein results in increased activation of T cells. In some examples, binding of the nanoparticle to both the target antigen and to a T cell results in activation of the T cell (e.g., increased cytokine production such as IL-2, IL-6, IL-10, IFN-γ, or TNF-α; increased surface marker expression such as CD69; or increased target cell lysis) and/or in improved killing of the target cell compared to T cells without contact with the nanoparticle composition.

Other features or advantages of the present invention will be apparent from the following drawings and detailed description of several examples, and also from the appended claims.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-C show schematics of exemplary nanoparticles of the disclosure. FIG. 1A: shows a schematic of a nanoBiTE with exemplary cancer cell targets and target antigens as well as exemplary size, polydispersity and Zeta potential. FIG. 1B: provides a schematic of a nanoMuTEs. FIG. 1C: shows a schematic of the BCMA/CD3, CS1/CD3, CD38/CD3 nanoBiTEs and BCMA/CS1/CD38/CD nanoMuTEs.

FIG. 2A-C include bar graphs showing the expression of various cell surface antigens on MM cells. FIG. 2A: is a bar graph showing the expression of BCMA, CS1 and CD38 in MM cell lines H929, MM.1S, OPM2, and RPMI-8226. FIG. 2B: is a bar graph showing the expression of BCMA, CS1 and CD38 in MM patient cells. FIG. 2C is a box plot showing the mRNA expression levels of BCMA, CS1, and CD38 in 559 MM patients.

FIG. 3A-B include bar graphs depicting the levels of liposomal binding to MM cells. FIG. 3A: is a bar graph showing nanoBiTE and nanoMuTE binding to H929, MM1.s, OPM2 and RPMI-8226 MM cell lines. FIG. 3B: is a bar graph showing the level nanoBiTE and nanoMuTE binding to MM cells isolated from 3 patients.

FIG. 4A-B show bar graphs depicting the level of liposomal binding in the presence of various blocking antibodies. FIG. 4A: is a bar graph showing the liposomal binding levels of various nanoBiTE compositions in the presence or absence of blocking antibodies. FIG. 4B: is a bar graph showing the liposomal binding levels of various nanoMuTE compositions in the presence or absence of blocking antibodies.

FIG. 5A-D include graphs showing the activation of in the presence of varying concentrations of nanoBiTE or nanoMuTE compositions. FIG. 5A: graphically depicts the percent CD4/CD69 positive cells. FIG. 5B: graphically depicts the percent CD8/CD69 positive cells. FIG. 5C: graphically depicts the levels of IL-2, IL-6, IL-10, IFN-gamma, and TNF-alpha secretions following a four-day incubation of MM and PBMCs incubated with Isotype/CD3, BCMA/CD3, CS1/CD3, or CD38/CD3 nanoBiTEs or BCMA/CS1/CD38/CD3 nanoMuTEs in the 3DTEBM. FIG. 5D shows bar graphs depicting the percent of CD69 positive CD4 and CD8 T cells culture with MM cells and various nanoBiTE and nanoMUTE compositions.

FIG. 6A-B include bar graphs showing T cell-induced MM cell lysis resulting from contact with various nanoBiTE or nanoMuTE compositions. FIG. 6A: is a bar graph depicting the survival (percent of untreated) of MM cell lines H929, MM.1S, OPM2, and RPMI-8226 in the presence or absence of nanoBiTEs or nanoMuTEs and T cells. FIG. 6B: is a bar graph showing T cell-induced MM cell lysis of primary samples in 3DTEBM.

FIG. 7A-D show a schematic and graphs of an in vivo study examining the effects associated with nanoBiTE administration. FIG. 7A: shows a timeline for the in vivo study. FIG. 7B: is a graph showing photon flux. FIG. 7C: is a graph showing the survival with and without CD38/CD3 nanoBiTEs.

FIG. 8A-D include graphs depicting nanoBiTE binding to various cancer cell types. FIG. 8A: is a bar graph showing liposomal binding of untreated, isotype/CD3 and CD38/CD3 to MM cell lines. FIG. 8B: is a bar graph showing liposomal binding of untreated, isotype/CD3 and CD33/CD3 to leukemia cell lines. FIG. 8C: is a bar graph showing liposomal binding of untreated, isotype/CD3 and CD20/CD3 to lymphoma cell lines. FIG. 8D: is a bar graph showing liposomal binding of untreated, isotype/CD3 and EpCAM/CD3 to MM cell lines.

FIG. 9A-C include graphs depicting the activation of CD8 and CD4 T cells to nanoBiTEs. FIG. 9A: shows the percent of CD69 positive CD8 T cells and CD4 T cells in untreated, isotype/CD3 treated and CD38/CD3 treated conditions in co-culture with MM cells. FIG. 9B: shows the percent of CD69 positive CD8 T cells and CD4 T cells in untreated, isotype/CD3 treated and CD20/CD3 treated conditions in co-culture with lymphoma cells. FIG. 9C: shows the percent of CD69 positive CD8 T cells and CD4 T cells in untreated, isotype/CD3 treated and CD33/CD3 treated conditions in co-culture with leukemia cells.

FIG. 10A-C include graphs depicting the nanoBiTE directed killing of cancer cells in vitro. FIG. 10A: is a bar graph showing survival of MM cells in culture with T cells and are untreated, isotype/CD3 treated or CD38/CD3 treated. FIG. 10B: is a bar graph showing survival of lymphoma cells in culture with T cells and are untreated, isotype/CD3 treated or CD20/CD3 treated. FIG. 10C: is a bar graph showing survival of leukemia cells in culture with T cells and are untreated, isotype/CD3 treated or CD33/CD3 treated.

FIG. 11 is a schematic showing the experimental design of the in vivo cancer killing experiments and shows the percent survival, photon flux and tumor progressing in mice injected with MM cells, PMBCs, and CD38/CD3 nanoBiTE or lymphoma cells, PMBCs and CD20/CD3 nanoBiTE.

FIG. 12 is a schematic showing the experimental design of the in vivo cancer killing experiments and shows the percent survival and tumor progressing in mice injected with MM cells, PMBCs, and BCMA/CS1/CD38/CD3 nanoMUTEs, isotype/CD3 nanoBiTE, BCMA/CD3 nanoBiTE, CS1/CD3 nanoBiTE, or CD38/CD3 nanoBiTE.

FIG. 13 is a graph showing the pharmokinetics of nanoBiTEs vs. traditional BiTEs.

### DETAILED DESCRIPTION

During the course of disease, cancers generally become more heterogeneous. The process of conversion from a nonmalignant to a malignant cell is understood to occur through the sequential acquisition of alterations that lead to enhanced cellular proliferation, evasion of growth suppression and cell death signals, induction of angiogenesis, and, ultimately, activation of programs leading to tissue invasion and metastasis. Even after malignant transformation, a cancer remains dynamic and continues to evolve. This ongoing evolution might ultimately generate a molecularly heterogeneous tumor consisting of cancer cells harboring distinct molecular signatures (e.g., distinct expression patterns of cell surface markers) with differential levels of sensitivity to anticancer therapies. This heterogeneity can result from genetic, transcriptomic, epigenetic, and/or phenotypic changes. This heterogeneity underlies resistance and site-specific responses and also complicates the selection of globally effective therapeutic agents. Even in the simplest scenario of an oncogene-driven cancer, heterogeneity ultimately provides the seeds for the emergence of resistance and, eventually, disease relapse.

The present disclosure is based, at least in part, in the development of a multi-specific nanoparticle T cell engager system. This system allows for efficient in targeting to heterogeneous cancer cells expressing a variety of heterotypic genes and moieties. In some embodiments, the nanoparticle system as disclosed herein is able to redirect endogenous immune cells to target cells. As demonstrated below in the specific examples, the nanoparticle system shows significantly greater binding to a variety of cancer cells and the ability to prompt T cell-induced cancer cell lysis relative to monovalent targeting nanoparticles in vitro experiments. Moreover, the system allows for efficient and low cost production as well as low toxicity risks. Accordingly, the present disclosure relates to compositions of a nanoparticle system, and methods of using a nanoparticle system to treat cancer (e.g., Multiple Myeloma). Compositions and methods of the nanoparticle delivery system are described below.

### I. Composition

Aspects described herein stem, at least in part, from development of a nanoparticle system comprising (a) at least one antigen binding moiety that specifically binds to an antigen present on a desired target cell and (b) at least one T cell moiety that specifically binds T cells. In some aspects, the nanoparticle system comprises (a) multiple antigen binding moieties (e.g., 2, 3, 4, or more) that specifically bind to different antigens on a desired target cell and (b) at least one T cell moiety that specifically binds T cells. In some embodiments, the composition comprises nanoparticles that have at least one antigen binding moiety, and at least one T cell moiety on the surface of the nanoparticles, and at least one active agent encapsulated in the nanoparticles.

The nanoparticle system as disclosed herein redirects T cells to the target cells, where the T cells mediate lysis of the target cells. As used herein the terms “engages T cells” or “redirects T cells” are used interchangeably and refer to the ability of the compositions of the disclosure to bring into close proximity T cells onto a defined antigen, and thereby target cells expressing the antigen, through binding to the nanoparticle. Thus, the disclosed compositions and methods do not rely on endogenous T cell recognition of target cells, but instead, binding of the nanoparticle to both the target antigen and to a T cell results in activation of the T cell (e.g., increased cytokine production such as IL-2, IL-6, IL-10, IFN-γ, or TNF-α; increased surface marker expression such as CD69; or increased target cell lysis) and in improved killing of the target cell. A composition of the present disclosure may also comprise a suitable pharmaceutically acceptable carrier known in the art. Components of nanoparticle system are described below.

(a) Nanoparticle

As used herein, the term nanoparticle refers to a particle that has a diameter of less than 1 um (1000 nm). Nanoparticles used in the system described herein can be made of metals such as iron, nickel, aluminum, copper, zinc, cadmium, titanium, zirconium, tin, lead, chromium, manganese and cobalt; metal oxides and hydrated oxides such as aluminum oxide, chromium oxide, iron oxide, zinc oxide, and cobalt oxide; metal silicates such as of magnesium, aluminum, zinc, lead, chromium, copper, iron, cobalt, and nickel; alloys such as bronze, brass, stainless steel, and so forth. Nanoparticles can also be made of non-metal or organic materials such as cellulose, ceramics, glass, nylon, polystyrene, rubber, plastic, or latex. In some embodiments, nanoparticles are formed from a combination of a metal and a non-metal or organic compound, for example, methacrylate- or styrene-coated metals and silicate-coated metals. The base material can be doped with an agent to alter its physical or chemical properties. For example, rare earth oxides can be included in aluminosilicate glasses to create a paramagnetic glass materials with high density (see White & Day, Key Engineering Materials Vol. 94-95, 181-208, 1994). Alternatively, nanoparticles can be made entirely of biodegradable organic materials, such as cellulose, dextran, and the like.

Suitable commercially available nanoparticles include, for example, nickel nanoparticles (Type 123, VM 63, 18/209A, 10/585A, 347355 and HDNP sold by Novamet Specialty Products, Inc., Wyckoff, N.J.; 08841R sold by Spex, Inc.; 01509BW sold by Aldrich), stainless steel nanoparticles (P316L sold by Ametek), zinc dust (Aldrich), palladium nanoparticles (D13A17, John Matthey Elec.), M-450 Epoxy Beads (Dynal), TiO₂, SiO₂, and MnO₂ nanoparticles (Aldrich); and IgG-coated beads available from Miltenyi Biotec.

In some embodiments, nanoparticles of the disclosure can be made from liposomes. As used herein, the terms “liposome” or “liposomal nanoparticle” comprise one or more lipid bilayers, wherein each layer is an amphiphilic lipid arranged to face each other it refers to a self-assembling structure comprising two monolayers containing molecules. Amphiphilic lipids comprise groups in which a polar (hydrophilic) head group is covalently linked to one or two or more nonpolar (hydrophobic) acyl or alkyl chains. The amphipathic lipid molecules are arranged such that the polar head groups are oriented towards the surface of the bilayer and the acyl chains are doubled, since the contact of the hydrophobic acyl chain with the surrounding aqueous medium is energetically unfavorable. Aligned toward the inside of the layer, effectively preventing the acyl chains from contacting the aqueous environment. Liposomes useful in combination with the methods and compositions described herein may be a single lipid bilayer (monolamellar liposome) or multiple lipid bilayers (multilamellar vesicles) enclosed or enclosed in an aqueous compartment. Various types of liposomes have been described, see, e.g., Cullis et al., Biochim. Biophys Acta, 559: 399-420 (1987).

Amphipathic lipids typically comprise the major structural elements of liposomal lipid vesicles. The hydrophilic character of lipids derives from the presence of phosphate, carboxyl, sulfate, amino, sulfhydryl, nitro and other similar polar groups. Hydrophobicity by including, without limitation, groups containing saturated and unsaturated long chain aliphatic hydrocarbon groups that may be substituted with one or more aromatic groups, alicyclic groups or heterocyclic groups can be granted. Examples of amphiphilic compounds are phosphoglycerides and sphingolipids, and representative examples thereof include phosphatidyl choline, phosphatidyl ethanolamine, phosphatidyl serine, phosphatidyl inositol, phosphatidic acid, phosphatidyl glycerol (phoasphatidyl glycerol), palmitoyl oleoyl phosphatidyl choline, and the like. These include lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine and egg sphingomyelin. Other lipids (e.g., sphingolipids and glycosphingolipids) are also useful in the methods and compositions provided herein. In addition, the amphipathic lipids described above may be mixed with other lipids such as triacylglycerols, sterols.

Liposomes may be comprised of a variety of different types of phospholipids having varying hydrocarbon chain lengths. The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes may be prepared by sonicating lipids in an aqueous solution, solvent injection, lipid hydration, reverse evaporation, or freeze drying by repeated freezing and thawing. As would be apparent to one of ordinary skill, all of the parameters that govern liposome formation may be varied. These parameters include, but are not limited to, temperature, pH, concentration and composition of lipid, concentration of multivalent cations, rate of mixing, presence of and concentration of solvent.

The configuration of nanoparticles can vary from being irregular in shape to being spherical and/or from having an uneven or irregular surface to having a smooth surface. Preferred characteristics of nanoparticles can be selected depending on the particular conditions under which they will be prepared and/or used. Nanoparticles may be of uniform or variable size. Particle size distribution can be conveniently determined, for example, using dynamic light scattering.

In some embodiments, nanoparticles have a mean particle diameter of 2-500 nm. Nanoparticles may be substantially spherical in shape and the diameter of a group of nanoparticles may be represented by the average diameter of the nanoparticles in the group.

In some embodiments, nanoparticles have a mean particle diameter of 90-100 nm, 90-125 nm, 90-150 nm, 90-200 nm, 90-300 nm, 90-400, 90-500 nm, 100-125 nm, 100-150 nm, 100-200 nm, 100-300 nm, 100-400, 100-500 nm, 125-150 nm, 125-200 nm, 125-300 nm, 125-400, 125-500 nm, 150-200 nm, 150-300 nm, 150-400, 150-500 nm, 175-200 nm, 175-300 nm, 175-400, 175-500 nm, 200-300 nm, 200-400, 200-500 nm, 300-400, 300-500 nm, or 400-500 nm. In some aspects, mean particle diameter of a nanoparticle of the present disclosure may be about 150 nm.

Protein molecules can be bound to nanoparticles by various means well-known in the art, including adsorption and covalent coupling. In some embodiments, antibodies are bound to nanoparticles coated with anti-immunoglobulin antibodies (e.g., IgG-coated beads available from Miltenyi Biotec). In other embodiments, nanoparticles can be conjugated with a coupling agent (e.g., streptavidin) and coupling agent-conjugated-nanoparticles are then mixed with tagged proteins of interest (e.g., biotinylated monocloncal targeting antibodies).

(b) Antigen Binding Moiety

In some embodiments, the nanoparticles disclosed herein comprises at least one antigen binding moiety. In other aspects, the nanoparticles comprise 2, 3, 4, 5, or more antigen binding moieties. In some embodiments, the nanoparticles comprise multiple antigen binding moieties each specific for a distinct target. As used herein, the term “antigen binding moiety” refers to a molecule that may bind to a specific molecule on a target, and that directs a nanoparticle to a particular location or cell. As described above, an antigen binding moiety may be attached to the surface of a nanoparticle through covalent, non-covalent, or other associations. Non-limiting examples of an antigen binding moiety may include synthetic compounds, natural compounds or products, macromolecular entities, and bioengineered molecules, and may include antibodies, antibody fragments, polypeptides, lipids, polynucleotides, and small molecules such as ligands, and hormones.

In an aspect, the antigen binding moiety may be an antibody. As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring).

Antibodies that specifically bind to antigens or epitopes present on the desired target cells are used to bring antigen-specific T cells in sufficient proximity to the target cells to effect killing of those cells.

Specific binding occurs to the corresponding antigen even in the presence of a heterogeneous population of proteins and other biologics. “Specific binding” of an antibody means that the binds to its target antigen with an affinity that is substantially greater than the antibody's binding to an irrelevant antigen. The relative difference in affinity is often at least 25% greater, more often at least 50% greater, most often at least 100%. The relative difference can be at least 2×, at least 5×, at least 10×, at least 25×, at least 50×, at least 100×, at least 1000×, for example.

Depending on the type of antibody employed, an antibody can be isolated, prepared synthetically, or genetically engineered, all using well-known techniques. See, e.g., US 2013/0034566 and US 2013/0028932, both of which are incorporated herein by reference in their entireties.

A nanoparticle can be directed to a variety of target cell types, including tumor cells, depending on the specificity of the antigen binding moiety on the nanoparticle. In some embodiments, the antigen binding moiety specifically binds to a tumor-associated antigen. Tumor-associated antigens include unique tumor antigens expressed exclusively by the tumor from which they are derived, shared tumor antigens expressed in many tumors but not in normal adult tissues (oncofetal antigens), and tissue-specific antigens expressed also by the normal tissue from which the tumor arose. Tumor-associated antigens can be, for example but not limited to, embryonic antigens, antigens with abnormal post-translational modifications, differentiation antigens, products of mutated oncogenes or tumor suppressors, fusion proteins, or oncoviral proteins.

A variety of tumor-associated antigens are known in the art, and many of these are commercially available. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastric carcinoma), alphafetoprotein receptor (expressed in multiple tumor types, particularly mammary tumors), and M2A (expressed in germ cell neoplasia).

Tumor-associated differentiation antigens include tyrosinase (expressed in melanoma) and particular surface immunoglobulins (expressed in lymphomas).

Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer), ras, p53, nonmutant associated with breast tumors, MAGE-1, and MAGE-3 (associated with melanoma, lung, and other cancers). Fusion proteins include, but are not limited to, BCR-ABL, which is expressed in chromic myeloid leukemia. Non limiting examples of oncoviral proteins include HPV type 16, E6, and E7, which are found in cervical carcinoma.

Non limiting examples of tissue-specific antigens include melanotransferrin and MUC 1 (expressed in pancreatic and breast cancers); CD10 (previously known as common acute lymphoblastic leukemia antigen, or CALLA) or surface immunoglobulin (expressed in B cell leukemias and lymphomas); the α chain of the IL-2 receptor, T cell receptor, CD45R, CD4+/CD8+ (expressed in T cell leukemias and lymphomas); prostate-specific antigen and prostatic acid-phosphatase (expressed in prostate carcinoma); GP 100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Non limiting examples of tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

Tissue-specific antigens include epithelial membrane antigen (expressed in multiple epithelial carcinomas), CYFRA 21-1 (expressed in lung cancer), Ep-CAM (expressed in pan-carcinoma), CA125 (expressed in ovarian cancer), intact monoclonal immunoglobulin or light chain fragments (expressed in myeloma), and the beta subunit of human chorionic gonadotropin (HCG, expressed in germ cell tumors).

In some embodiments, non-limiting examples of suitable targets may include cells of MM, acute myeloid leukemia, non-Hodgkin's lymphoma, chronic lymphocytic leukemia, colorectal cancer, or non-small-cell lung cancer. Non-limiting examples of additional targets may include CD38, CD56, CD138, CD20, CD52, CD33, CD20, epidermal growth factor receptor, epithelial cell adhesion molecule (EpCAM), human epidermal growth factor receptor, lewis antigen, or carcinoembryonic antigen. In one aspect, nanoparticles of the present disclosure may target cells expressing CD38, CS1, CD20, CD33, EpCAM and/or B Cell Maturation Antigen (BCMA), such as MM cells, lymphoma cells, leukemia cells, lung cancer cells, colon cancer cells, breast cancer cells, and/or prostate cancer cells.

As used herein “target” refers to a property of the nanoparticles of the disclosure, to home to and bind specific cells of interest that may express a specific molecule, such as an antigen, to which an antigen binding moiety associated with a nanoparticle binds.

As used herein, “target cell” refers to the cell of interest to which a targeting molecule of a nanoparticle of the present disclosure binds. The term “target” also encompasses a cell or protein of interest to which delivery of a T cell population is desired.

(c) T Cell Moiety

In some embodiments, the nanoparticles disclosed herein comprise at least one T cell moiety. As used herein, the term “T cell moiety” refers to a molecule that specifically binds to and/or activates a specific molecule on expressed on T cells, such as CD3, in order to redirect and/or active them to a desired target, using a nanoparticle as described above. The “antigen-specificity” of a T cell refers to the fact that the T cells are subpopulations, e.g., subpopulations of highly effective cytotoxic T cells specific for, e.g., a viral antigen or an antigen from another pathogen, or subpopulations of helper T cells. Several types of moieties can be used for this purpose.

In some embodiments, the moiety is an anti-TCR-specific antibody, such as an antibody that specifically binds to a TCR present on a population of T cells.

In some embodiments, the moiety is an MHC class I-immunoglobulin complex, an MHC class I molecule (e.g., a soluble monomer or multimer), an MHC class II molecule (e.g., a soluble monomer or multimer), or an MHC class II-immunoglobulin complex. Such moieties comprise an antigenic peptide to which the T cell is directed.

In some embodiments, the moiety is an MHC class I-immunoglobulin complex comprising (i) an immunoglobulin molecule comprising two immunoglobulin heavy chains and two immunoglobulin light chains; and (ii) two MHC class I molecules, each comprising an a chain and a β2 microglobulin. Each α chain comprises α1, α2, and α3 domains, and the α1 and α2 domains of each α chain form a peptide binding cleft. The N terminus of each immunoglobulin heavy chain is linked to the N terminus of each α3 domain, and the peptide binding cleft comprises an antigenic peptide recognized by the T cell. Such complexes and their production are described in U.S. Pat. No. 6,268,411, which is incorporated herein by reference in its entirety.

In some embodiments, the moiety is an MHC class I molecule comprising an antigenic peptide recognized by the T cell. In some embodiments, the MHC class I molecule is a soluble monomeric form. In some embodiments, the MHC class I molecule is a soluble multimeric form. See, e.g., U.S. Pat. No. 7,074,905, which is incorporated herein by reference in its entirety.

In some embodiments, the moiety is an MHC class II molecule comprising an antigenic peptide recognized by the T cell. In some embodiments, the MHC class II molecule is a soluble monomeric form. In some embodiments, the MHC class II molecule is a soluble multimeric form. See, e.g., U.S. Pat. No. 7,074,905, which is incorporated herein by reference in its entirety.

In some embodiments, the moiety is an MHC class II-immunoglobulin complex comprising four fusion proteins. Two first fusion proteins comprise (1) an immunoglobulin heavy chain, and (2) an extracellular domain of an MHC class II chain; and two second fusion proteins comprise (1) an immunoglobulin light chain and (2) an extracellular domain of an MHC class II α chain. The fusion proteins associate to form the molecular complex, which comprises two ligand binding sites, each ligand binding site formed by the extracellular domains of the α and β chains. Such complexes and their production are described in U.S. Pat. No. 6,015,884, which is incorporated herein by reference in its entirety.

In an aspect, a nanoparticle delivery system of the present disclosure may be used to deliver an active agent to a cell or site of interest. In an aspect, a nanoparticle of the disclosure may encapsulate an active agent. As used herein “active agents” may be therapeutic agents, diagnostic agents, or a combination thereof. Non-limiting examples of an active agent may include proteasome inhibitors, histone deacetylase inhibitors, chemotherapeutic agents, immunomodulating agents, or other agents that may be toxic to or kill cancer cells. Non-limiting examples of proteasome inhibitors may include bortezomib, carfilzomib, marizomib, ixazomib, or MLN9708. Non-limiting examples of a histone deacetylase inhibitor may be panobinostat, vorinostat, zolinza, romidepsin, or Istodax. Non limiting examples of chemotherapeutic agents may be doxorubicin, melphalan, vincristine, cyclophosphamide, etoposide, or bendamustine. Non-limiting examples of immunomodulating agents may be thalidomide, lenalidomide, or pomalidomide.

A nanoparticle of the disclosure may carry an active agent. The active agent, which may be a therapeutic agent may be associated with the surface of, encapsulated within, surrounded by, or dispersed throughout the nanoparticle. In a preferred aspect of the disclosure, an active agent is encapsulated within the core of a nanoparticle.

A pharmaceutical composition of the invention may also comprise one or more nontoxic pharmaceutically acceptable carriers, adjuvants, excipients, and vehicles as desired. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with a nanoparticle of the invention, use thereof in the compositions is contemplated. Supplementary active compounds may also be incorporated into the compositions.

A pharmaceutical composition of the invention may be formulated to be compatible with its intended route of administration. Suitable routes of administration may include parenteral, oral, pulmonary, transdermal, transmucosal, and rectal administration. The term parenteral, as used herein, includes subcutaneous, intravenous, intramuscular, intrathecal, or intrasternal injection, or infusion techniques.

Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

In preferred embodiments, a pharmaceutical composition of the invention is formulated to be compatible with parenteral administration. For instance, pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.), or phosphate buffered saline (PBS). In exemplary embodiments, a pharmaceutical composition of the invention is formulated with phosphate buffered saline (PBS).

In all cases, a composition may be sterile and may be fluid to the extent that easy syringe ability exists. A composition may be stable under the conditions of manufacture and storage, and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Systemic administration may also be by transmucosal or transdermal means. For transmucosal or transdermal administration, penetrants appropriate to the barrier to be permeated are used in the formulation. Such penetrants are generally known in the art, and may include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds may also be prepared in the form of suppositories (e.g., with conventional suppository bases such as cocoa butter and other glycerides) or retention enemas for rectal delivery.

In one embodiment, a nanoparticle of the present invention may be prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

Additional formulations of pharmaceutical nanoparticle compositions may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the nanoparticles would be apparent to one of ordinary skill in the art.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### II. Methods

Also provided by the disclosure is a method of treating cancer in a subject in need by administration of a therapeutically effective amount of a composition comprising the nanoparticle system as described herein, so as to recognize an antigen on cancer cells and the engage T cells, thereby reducing cancer progression. The therapeutically effective amount can also redirect T cells to tumor cells which results in an efficacious immune response (e.g., activation of T cells) against a tumor and/or produce improved tumor cell killing for heterogeneous tumors.

In some embodiments, the nanoparticle system as disclosed herein has an in vivo half-life of at least 10 hours, at least 15 hours, at least 20 hours, at least 25 hours, at least 30 hours, at least 35 hours, at least 40 hours, at least 50 hours, at least 55 hours, at least 60 hours, at least 65 hours, at least 70 hours, at least 75 hours, at least 80 hours, at least 85 hours, at least 90 hours, at least 95 hours. In an exemplary embodiment, the nanoparticle system of the disclosure has an in vivo half-life of 60 hours.

In an aspect, the present invention encompasses administering a therapeutically effective amount of a nanoparticle composition to a subject in need thereof. As used herein, the phrase “a subject in need thereof” refers to a subject in need of preventative or therapeutic treatment. A subject may be a rodent, a human, a livestock animal, a companion animal, or a zoological animal. In one embodiment, a subject may be a rodent, e.g., a mouse, a rat, a guinea pig, etc. In another embodiment, a subject may be a livestock animal. Non-limiting examples of suitable livestock animals may include pigs, cows, horses, goats, sheep, llamas and alpacas. In still another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In a preferred embodiment, a subject is a mouse. In another preferred embodiment, a subject is a human.

A nanoparticle composition of the invention is formulated to be compatible with its intended route of administration. Suitable routes of administration include parenteral, oral, pulmonary, transdermal, transmucosal, and rectal administration. In preferred embodiments, a pharmaceutical composition of the invention is administered by injection.

One of skill in the art will recognize that the amount and concentration of the composition administered to a subject will depend in part on the subject and the reason for the administration. Methods for determining optimal amounts are known in the art. Generally, a safe and effective amount of a nanoparticle composition is, for example, that amount that would cause the desired therapeutic effect in a subject while minimizing undesired side effects. In various embodiments, an effective amount of a nanoparticle composition described herein can substantially inhibit cancer progression, slow the progress of cancer, or limit the development of cancer.

Compositions of the invention are typically administered to a subject in need thereof in an amount sufficient to provide a benefit to the subject. This amount is defined as a “therapeutically effective amount.” A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

When used in the treatments described herein, a therapeutically effective amount of a nanoparticle composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to recognize an antigen on cancer cells and the engage T cells, reducing cancer progression.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shawl (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a nanoparticle composition can occur as a single event or over a time course of treatment. For example, a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities cancer.

A nanoparticle composition can be administered simultaneously or sequentially with another agent, such as an antibiotic, an anti-inflammatory, or another agent. For example, a nanoparticle composition can be administered simultaneously with another agent, such as an antibiotic or an anti-inflammatory.

In preferred aspects, a method of the invention is used to treat a neoplasm or cancer. The neoplasm may be malignant or benign, the cancer may be primary or metastatic; the neoplasm or cancer may be early stage or late stage. A cancer or a neoplasm may be treated by delivering nanoparticles carrying a therapeutic agent to at least one cancer cell in a subject. The cancer or neoplasm may be treated by slowing cancer cell growth or killing cancer cells.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 jam), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

In some aspects, the nanoparticle delivery system of the disclosure may treat a cancer or a neoplasm by delivering a therapeutic nanoparticle to a cancer cell in a subject in vivo. Non-limiting examples of neoplasms or cancers that may be treated with a method of the invention may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood). In a preferred embodiment, a method of the invention may be used to treat T-cell leukemia and lymphoma. In an exemplary embodiment, a method of the disclosure is used to treat MM in a subject.

In other aspects, a nanoparticle delivery system of the disclosure may deliver a therapeutic nanoparticle to a cancer cell in vitro. A cancer cell may be a cancer cell line cultured in vitro. In some alternatives of the embodiments, a cancer cell line may be a primary cell line that is not yet described. Methods of preparing a primary cancer cell line utilize standard techniques known to individuals skilled in the art. In other alternatives, a cancer cell line may be an established cancer cell line. A cancer cell line may be adherent or non-adherent, or a cell line may be grown under conditions that encourage adherent, non-adherent or organotypic growth using standard techniques known to individuals skilled in the art. A cancer cell line may be contact inhibited or non-contact inhibited.

In some embodiments, the cancer cell line may be an established human cell line derived from a tumor. Non-limiting examples of cancer cell lines derived from a tumor may include the MM cell lines MM.1S, H929, and RPMI, osteosarcoma cell lines 143B, CAL-72, G-292, HOS, KHOS, MG-63, Saos-2, or U-2 OS; the prostate cancer cell lines DU145, PC3 or Lncap; the breast cancer cell lines MCF-7, MDA-MB-438 or T47D; the myeloid leukemia cell line THP-1, the glioblastoma cell line U87; the neuroblastoma cell line SHSYSY; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8, HCT 116, NCI-H716, NCI-H747, NCI-H508, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B or SW480; B16-F10, RAW264.7, the F8 cell line, or the pancreatic carcinoma cell line Panc1. In an exemplary embodiment, a method of the disclosure may be used to contact a cell of a MM cell line.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a nanoparticle composition described herein or components of a nanoparticle system as described herein. For example, liposomal nanoparticles (e.g., liposomes comprising cholesterol, DPPC, and DSPE), anti-CD3, anti-BCMA, anti-CS1, or anti-CD38. As another example, the components can be conjugated with biotin or avidin. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### EXAMPLES

### Example 1: Nanoparticle Multi-Specific T Cell Engagers for the Treatment of Multiple Myeloma

The goal of this study was develop a nanoparticle multi-specific T cell engager systems which overcome the limitations associated with monovalent immunotherapy regimens. While the understanding of molecular mechanisms including cell-signaling pathways and the interaction between MM cells and the bone marrow (BM) microenvironment has led to the development of novel therapies [Nair et al., Adv Pharmacol, 65: p. 143-89 (2012); de la Puente et al., Drugs Today (Barc), 49(9): p. 563-73 (2013)], which significantly improve the survival of MM patients [Kumar et al., Blood, 111(5): p. 2516-20 (2008); Mitsiades et al., Best Pract Res Clin Haematol, 20(4): p. 797-816 (2007)], the majority of MM patients still relapse due to the lack of complete efficacy in traditional therapies [Nair et al., Adv Pharmacol, 65: p. 143-89 (2012); Lobo et al., Annu Rev Cell Dev Biol, 23: p. 675-996 (2007); Ludwig et al., Leukemia, 27(1): p. 213-9 (2013)]. Therefore, there is an urgent need to develop novel therapeutic strategies for MM.

T cell-based immunotherapy such as chimeric antigen receptor (CAR)-T cells is an emerging strategy in the treatment of cancer [Lim et al., Cell, 168(4): p. 724-740 (2017)], particularly MM. CAR-T cell therapy uses autologous T cells to redirect them to a specific tumor antigen using genetic engineering of viral vectors. Several MM studies have showed promising preclinical [Drent et al., Haematologica, 101(5): p. 616-25 (2016); Nagle et al., Cancer J, 22(1): p. 27-33 (2016); Chen et al., Leukemia, 32(2): p. 402-412 (2018)] and clinical results [Ali et al., Blood, 128(13): p. 1688-700 (2016)] with the use of CAR-T cell technology. The CAR-T cells mostly pursue BCMA, CS1, or CD38 as antigens to target MM due to their high expressions and significant roles in the progression of MM [Chillemi et al., Front Biosci (Landmark Ed), 19: p. 152-62 (2014); Tai et al., Immunotherapy, 7(11): p. 1187-99 (2015)]. Problems with this technology relative to traditional therapies include toxicity and long-term safety profile of the viral vector, the need to perform quality control testing frequently throughout the production of CAR-T cells, the high costs associated with this technique due to the need of extensive labor and expensive facility equipment, and the inability to target multiple tumor antigens with one CAR-T cell [Zhang et al., Biomark Res, 5: p. 22 (2017)].

In addition to CAR T-cells, T cell-based therapy can be pursued with bispecific T-cell engagers (BiTEs). BiTE molecules are constructed from two different recognition domains (antibodies) linked through a protein linker, which still retains binding activity [Huston et al., Methods Enzymol, 203: p. 46-88 (1991); Mallender et al., J Biol Chem, 269(1): p. 199-206 (1994)]. One of the domains recognizes a tumor-associated surface antigen, while the other domain (antibody) recognizes T cells (via a receptor such as CD3) in order to activate and redirect cytotoxic T cells to tumor cells. BiTEs demonstrate extraordinary potency and efficacy against tumor cells [Wolf et al., Drug Discov Today, 10(18): p. 1237-44 (2005)], and do not need genetic engineering and manipulation of the patient's T cells, which overcomes some of the limitations of the CAR-T cell technology. One of the disadvantages of BiTEs, however, include the need to be recombinantly produced, which may hinder up-scale production.

One common theme between the CAR-T cell and BiTEs technologies is the ability to only target a single epitope on the cancer cell, while it is evidently known that cancer cells express a landscape of heterotypic genes and moieties [Bolli et al., Nat Commun, 5: p. 2997 (2014)]. This may cause these technologies to only affect some parts of the tumor and not others. To circumvent the impediment of only targeting one cancer moiety, recent studies have shown the targeting of multiple epitopes on the CAR-T cells or trispecific T cell engagers (2 cancer epitopes with 1 T cell epitope), but these approaches are still technically very challenging [Chen et al., Leukemia, 32(2): p. 402-412 (2018); Huston et al., Methods Enzymol, 203: p. 46-88 (1991)].

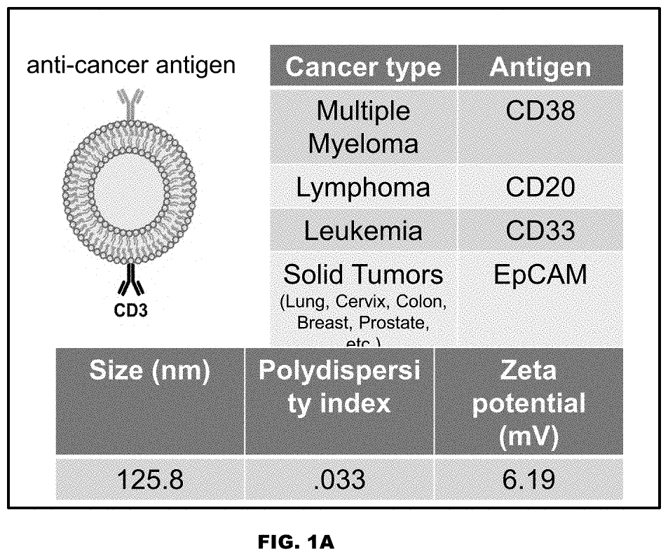

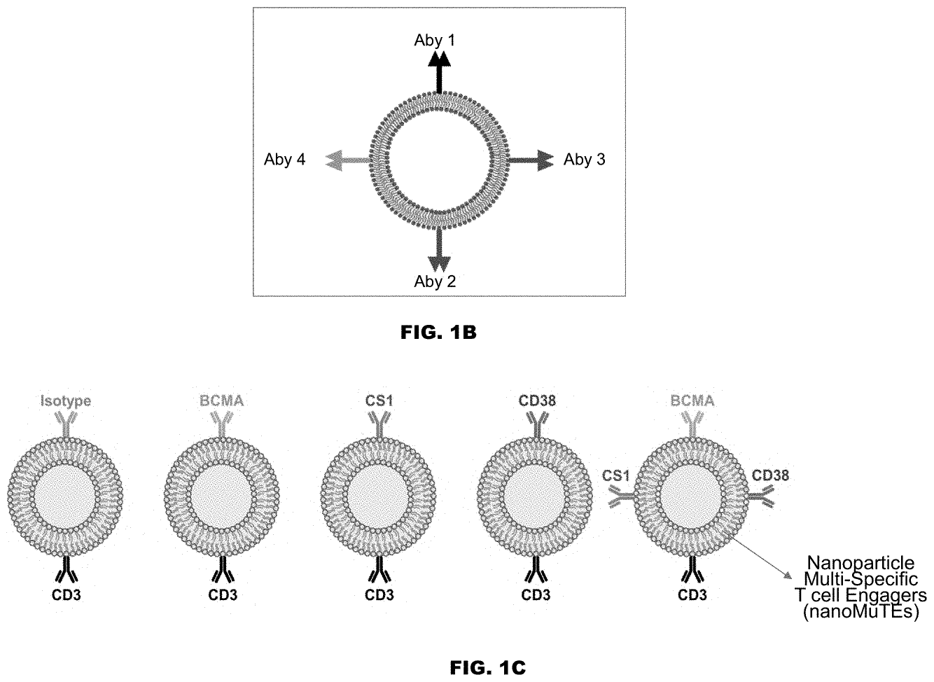

To overcome this difficulty, the present study provides a nanoparticle system in which two antibodies are chemically conjugated to the surface of a nanoparticle; one to recognize an epitope on MM cells and the other to engage T-cells called the Nanoparticle Bispecific T cell Engager (NanoBiTE; FIG. 1A). The main advantage of the NanoBiTE is due to the ease of production and the lack of creating genetically engineered autologous T cells. Moreover with the use of the same technological aspects, a NanoBiTE with three or more total targeting moieties called the Nanoparticle Multivalent T cell Engager (NanoMuTE; FIG. 1B). This gives a clear advantage to overcome the limitations of the CAR-T cell and classic BiTE technologies.

Taken together, the instant study has provided a nanoparticle system to redirect T cells to heterogeneous cancer cells with various moieties and reduce toxic side effects for effective tumor treatment. The results reported herein provide new therapeutic strategies for targeted treatment of MM.

### Methods

### Generation and Physicochemical Properties of the NanoBiTEs and NanoMuTE

Liposomal nanoparticles (liposomes) are developed by mixing three components: Cholesterol, DPPC, and DSPE—at a molar ratio of 3:6.5:0.5, respectively. The three components are solubilized in chloroform and the solution placed on a rotary-evaporator to develop a lipid film. The film is then hydrated with phosphate buffer solution (PBS) to form large multilamellar vesicles, which are downsized by extrusion. Finally, the liposomes are conjugated with 250 μg of streptavidin (Abcam) following the protocol of the manufacturer. The avidin-conjugated-liposome are then mixed overnight with or without biotinylated monocloncal targeting antibodies (e.g., antiCD3 and/or antiCD38) to develop the NanoBiTEs. The NanoBiTEs are characterized using dynamic light scattering (DLS; Malvern) to find the size, zeta potential, and polydispersity index (PDI). The stability of the NanoBiTEs are tested by incubation at 4° C., 20° C. (room temperature), and 37° C. for 24 hours, 3 days, 1 week, 1 month, and 6 months, then the NanoBiTEs are be analyzed by DLS.

### Binding of the NanoBiTEs and NanoMuTE to MM and T Cells In Vitro

The binding affinity of NanoBiTEs are tested for MM cell lines (H929, U266, MM1s, OPM2, and RPMI) and MM patient samples (n=5), as well as Jurkat cells and primary T cells by treating each cell type with DiD-labeled (red fluorescence) liposomes conjugated with: (1) Non-Targeted liposomes (liposomes lacking antibody conjugation) or untreated (no liposome control); (2) Single Targeted liposomes (e.g., only 1 targeting antibody, such as antiCD3, or a combination of targeting antibody and a non-specific antibody); (3) NanoBiTE liposomes (two distinct targeting antibodies, e.g., antiCD3/antiCD38-NanoBiTEs); or (4) NanoMuTE liposomes (at least 3 distinct targeting antibodies, e.g., antiCD3/antiCD38/antiCS1/antiBCMA). Cells are incubated with each of the four formulations (1 mg/mL) for four hours, then the cells are washed and analyzed by flow cytometry for the fluorescence intensity of DiD.

### Biodistribution and Pharmacokinetics of NanoBiTEs In Vivo

Due to the need of indigenous immune cells in mice, KaLwRij mice are used, an immune-competent mouse which allows growth of murine MM cell line 5TGM. 25 KaLwRij mice are each injected with one million 5TGM1 cells tagged with green fluorescent protein and luciferase (5TGM1-GFP-Luc) to enable tracking of the MM cells via bioluminescent imaging (BLI) and flow cytometry. Three weeks following 5TGM1-GFP-luc cell injection, tumor development is confirmed with BLI; once the tumors have developed, the mice are randomly split into 5 groups of 5 mice each, which will be treated with: (1) Vehicle; (2) non-targeted (no antiCD3 or antiCD38) liposomes; (3) antiCD3-Targeted liposomes (no antiCD38); (4) CD38-targeted liposomes (no antiCD3); and (5) antiCD3/antiCD38-NanoBiTEs (1 mg/kg once). The liposomes in all the groups will be labeled with DiD. Twenty four hours later, the organs are extracted (BM, blood, liver, spleen, lungs, and kidneys), and single cell suspensions from each organ developed and stained with V450-anti-CD3. Cells are then analyzed using flow cytometry for the amount of MM cell (GFP+) and infiltrating T Cells (V450+) in each organ, as well as the mean fluorescence intensity (MFI) of each of these cells of DiD which will reflect the binding of liposomes to these cells.

### Therapeutic Efficacy of the NanoMuTEs In Vivo

Sixty SCID mice are injected with a mixture of multiple MM cell lines (H929-Luc, RPMI-Luc and OPM2-Luc, 2.5×10\vec{ }⁵ cells each, a total of 1×10\hat{ }⁶ cell per each mouse). Two weeks following the MM cell injection, the mice are randomly split into 6 groups of 10 mice each, which are treated with: (1) Vehicle; (2) 10\hat{ }⁷ cells human-activated T cells once a week (3) 10\hat{ }⁷ cells human-activated T cells and next day antiCD3/antiCD38 NanoBiTE (1 mg/kg); (4) 10\hat{ }⁷ cells human-activated T cells and next day antiCD3/antiBCMA NanoBiTE (1 mg/kg); (5) 10\hat{ }⁷ cells human-activated T cells and next day antiCD3/antiCS1 NanoBiTE (1 mg/kg); and (6) 10\hat{ }⁷ cells human-activated T cells and next day antiCD3/antiCD38/antiBCMA/antiCS1 NanoMuTEs (1 mg/kg). Mice are treated once a week for 4 weeks; and followed for tumor progression using BLI once a week, and for survival daily, for 4 weeks.

### Results

(i) Significantly Greater Binding is Induced by nanoMuTEs Compared to Each Individual nanoBiTE

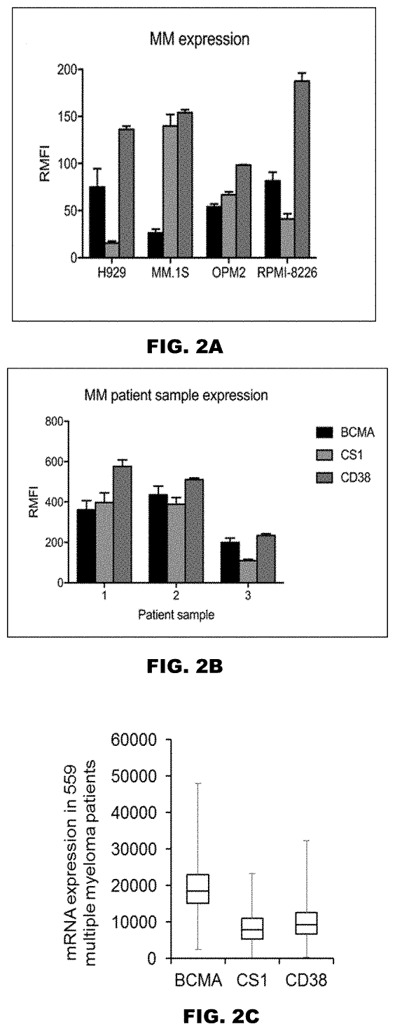

To elucidate the expression patterns of various cell surface antigens on MM cells four different immortal MM cell lines as well as primary MM cells isolated from patients we tested for the presence of BCMA, CS1 and CD8 antigens. Across all four cell lines, there is not two that have the same expression levels for a single antigen (FIG. 2A). These finding are consistent with the noted pitfalls of traditional monovalent immunotherapy regimens in the treatment of MM, specifically the inability to engage more than one target while MM presents an overwhelmingly large and dynamic repertoire on the cell surface [Zhang et al., Biomark Res, 5: p. 22 (2017)]. For instance, treating H929 with a CS1 BiTE or CAR-T would not suffice due to the very low/negligible expression level of CS1. Likewise, the same idea applies to the MM patient cells shown in FIG. 2B. The ability of the therapeutic to engage the target and induce cell lysis would be significantly increased if multiple antigens are targeted even in the presence of low antigen expression levels as seen for Patient 3.

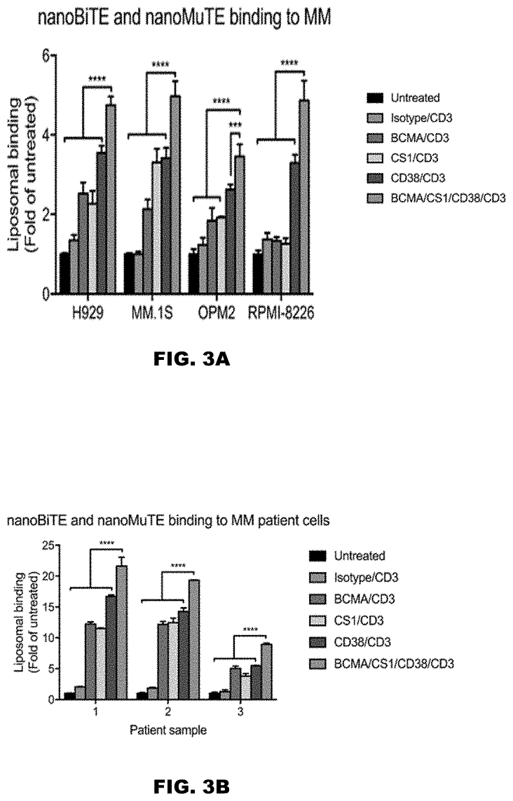

To assess the engagement of the nanoparticles to the cell receptor, the relative binding ability of the BCMA/CS1/CD38/CD3 nanoMuTEs to the isotype/CD3, BCMA/CD3, CS1/CD3, and CD38/CD3 nanoBiTEs was compared on MM cell lines and primary patient samples. The nanoMuTEs and nanoBiTEs were stained with a lipophilic tracer, DiD, and incubated with the cells for two hours prior to flow cytometry analysis. Across all four cell lines, the BCMA/CS1/CD38/CD3 nanoMuTEs bound significantly more to the MM compared to all other nanoBiTEs (FIG. 3A). Of course, the ability for the nanoMuTEs and nanoBiTEs to engage the BCMA, CS1, and CD38 receptors are reliant upon the expression levels of these antigens presented on the cell surface; hence the varying levels of bound particles. FIG. 3B exhibits the nanoMuTEs and nanoBiTEs engaged onto the same three patient primary samples that were shown in FIG. 2B. Similar to the observations seen in the MM cell lines, the BCMA/CS1/CD38/CD3 nanoMuTEs were significantly bound to the primary MM cells compared to the bivalent nanoBiTE particles.

(ii) The Specific Engagement of the nanoMuTEs and nanoBiTEs to MM is Receptor-Mediate.

Based on the results which found that the BCMA/CS1/CD38/CD3 nanoMuTEs bound significantly more to MM than the nanoBiTEs, the specificity to ensure proper receptor-mediated binding was investigated. The noted cells were incubated with 5 μg/mL of respective antibody prior to nanoBiTE and nanoMuTE incubation.

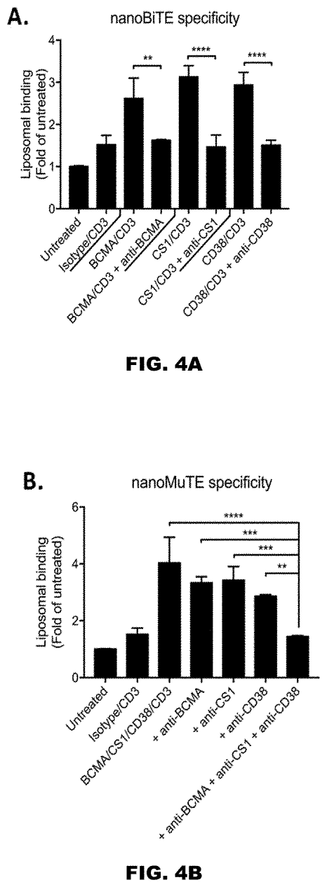

When anti-BCMA, CS1, or CD38 antibodies were incubated with OPM2 for one hour prior to BCMA, CS1, or CD38 nanoBiTEs, the binding of the nanoBiTEs were significantly decreased in comparison to the no addition of antibody (FIG. 4A). These quantitative values do not appear to be distinguishable from the values seen following the incubation of the isotype/CD3 nanoBiTEs. With regards to the BCMA/CS1/CD38/CD3 nanoMuTEs, no significant decrease of relative binding after the incubation of each individual antibody were observed (FIG. 4B); however, the binding of the BCMA/CS1/CD38/CD3 nanoMuTEs was identical to the isotype/CD3 nanoBiTEs following the incubation of antibodies targeting all three moieties.

(iii) CD4 and CD8 T Cell Activation is Only Induced by nanoBiTEs and nanoMuTEs in the Presence of the Target Cell.

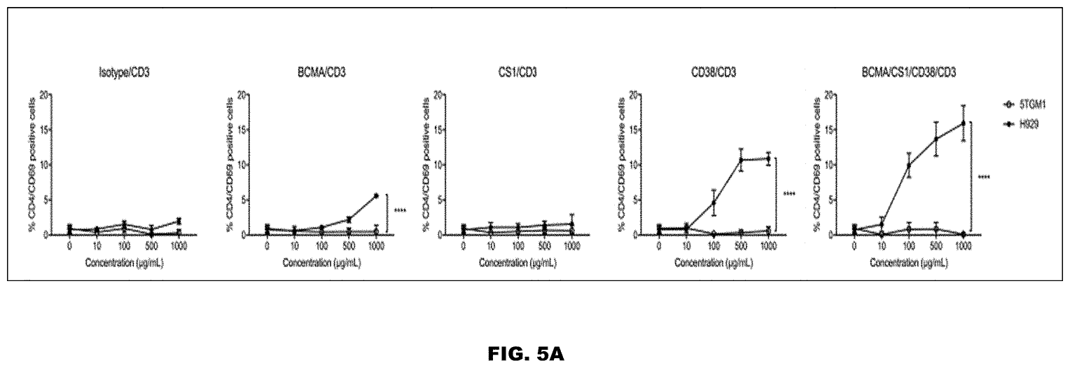

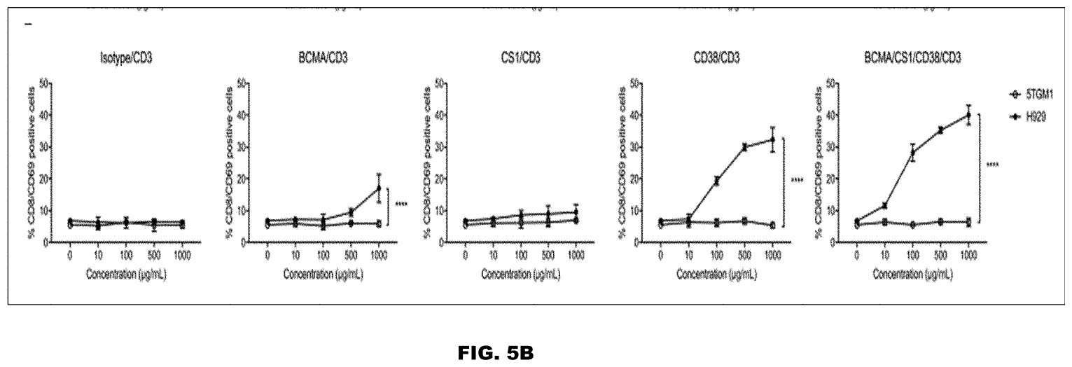

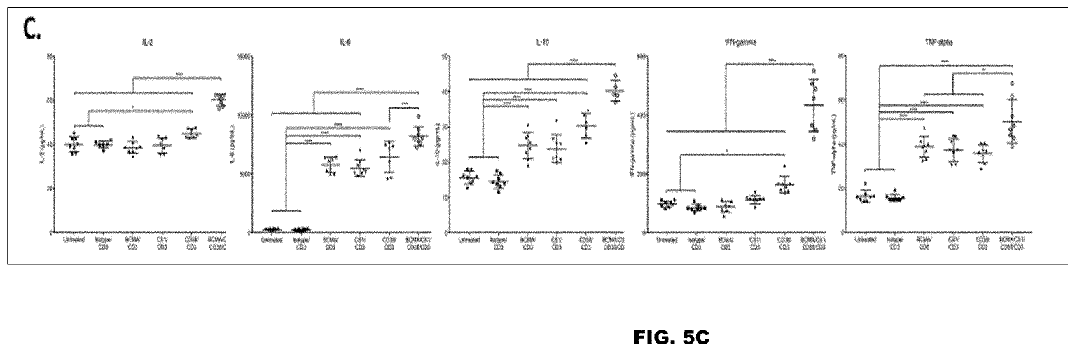

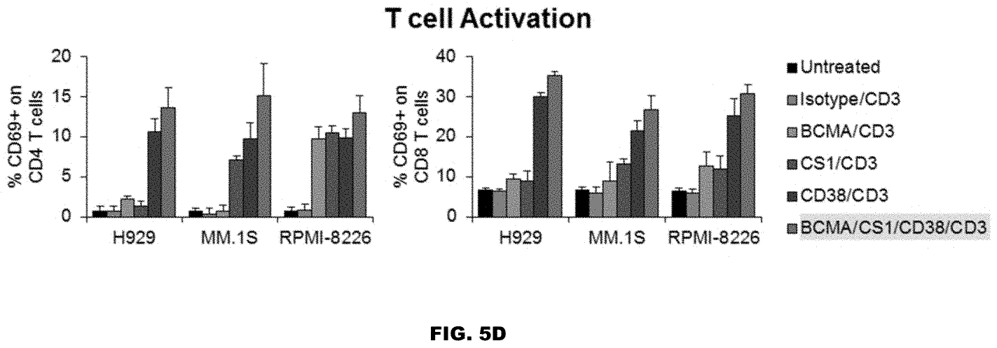

Next, the ability of the nanoMuTEs and various nanoBiTEs to induce T cell activation was tested. Previous preclinical studies have shown that both T cell-centered immunotherapies, BiTEs and CAR-T cells, only activate T cells in the presence of target-expressing cells; to successfully compare BiTE and CAR-Ts to the nanoparticle systems as disclosed herein, H929 or 5TGM1 cell lines are incubated with PBMCs and increasing concentrations of BCMA, CS1, or CD38 nanoBiTEs or BCMA/CS1/CD38/CD3 nanoMuTEs in the 3DTEBM. Following four days of incubation, CD4 and CD8 T cells were stained for CD69 and plotted in FIG. 5A and FIG. 5B. BCMA/CD3 and CD38/CD3 nanoBiTEs were able to enable an upregulation of CD69 in both CD4 and CD8 T cells only in the presence of H929 as concentration increased from 10 to 1000 μg/mL. The BCMA/CS1/CD38/CD3 nanoMuTEs enhanced the activation of both subtypes of T cells compared to the BCMA/CD3 and CD38/CD3 nanoBiTEs. T cell activation was not seen for the CS1/CD3 nanoBiTE due to the low CS1 expressions seen for H929 in FIG. 2A. In general, the percentage of activated CD8 T cells was approximately double the percentage seen for the activated CD4 T cells. No cross-reactivity of the nanoBiTEs and nanoMuTEs between different species was seen as shown by the use of 5TGM1. This further supports the notion that the nanoparticles as disclosed herein only activate T cells in the presence of target-expressing cells. With regards to the cytokines secreted by the T cells, the 3DTEBM cultures with H929 and the nanoBiTEs or nanoMuTEs were digested and dissolved for cytokine analysis shown in FIG. 5C. The cytokines most associated with T cell activation (IL-2, IL-6, IL-10, IFN-γ, and TNF-α) were measured. Each individual nanoBiTE was able to significantly induce the secretions of IL-6, IL-10, and TNF-α compared to the untreated and isotype/CD3 nanoBiTE conditions. IL-2 and IFN-γ were additionally upregulated by the CD38/CD3 nanoBiTEs. Finally, the BCMA/CS1/CD38/CD3 nanoMuTEs were able to manifest a significant increase in the cytokines, IL-2, IL-6, IL-10, IFN-γ, and TNF-α, compared to the untreated and all nanoBiTE conditions.

(iv) The nanoBiTEs and nanoMuTEs were Able to Prompt T Cell-Induced MM Cell Lysis In Vitro

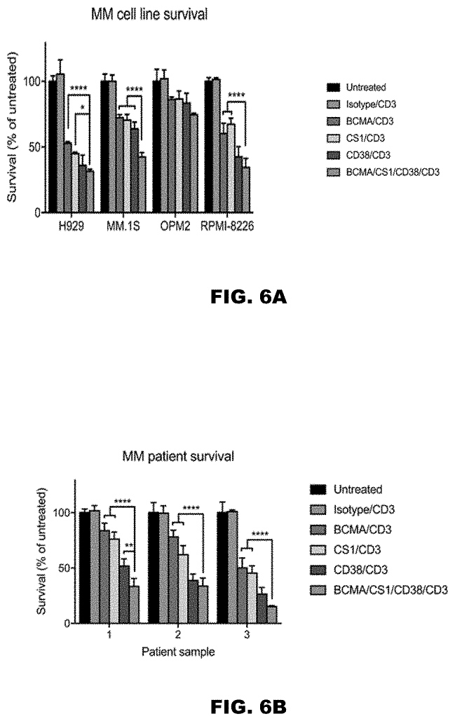

The role of the nanoBiTEs and nanoMuTEs to redirect T cells for the elimination of MM tumor cells was further examined. Each of the four DiO-stained MM cell lines and PBMCs at an E:T ratio of 2:1, respectively, were incubated with 500 μg/mL of the isotype/CD3, BCMA/CD3, CS1/CD3, or CD38/CD3 nanoBiTEs or the BCMA/CS1/CD38/CD3 nanoMuTEs in the 3DTEBM for four days. Afterwards, the number of live DiO-positive cells were counted and normalized by the number of counting beads. In FIG. 6A, each of the nanoBiTE and nanoMuTE regimen significantly increased MM cell lysis compared to the untreated and isotype/CD3 conditions. In addition, the BCMA/CS1/CD38/CD3 nanoMuTEs prompted significantly greater T cell-induced MM cell death than the BCMA/CD3 and CS1/CD3 nanoBiTEs in H929 and RPMI-8226, and compared to all three types of nanoBiTEs in the cell line, MM.1S. The same procedures were conducted for the findings of survival in primary MM cells (FIG. 6B); however instead of staining with DiO, the MM cells were stained with CD38 (APC) and CD3, CD14, CD16, CD19, CD123 (all FITC). Following flow analysis, a significant increase in primary MM cell lysis when Patient 1 cells was observed and PBMCs were incubated with BCMA/CS1/CD38/CD3 nanoMuTEs compared to using only each individual nanoBiTE. For Patients 2 and 3, T cells were redirected and induce MM cell death by the BCMA/CS1/CD38/CD3 nanoMuTEs significantly greater than the BCMA/CD3 and CS1/CD3 nanoBiTEs.

(v) The nanoBiTEs and nanoMuTEs were Able to Prompt T Cell-Induced MM Cell Lysis In Vivo

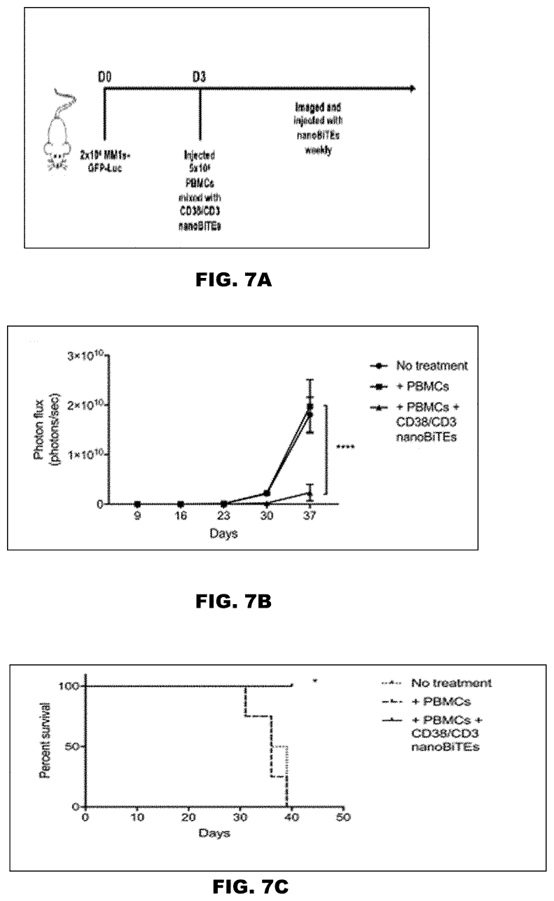

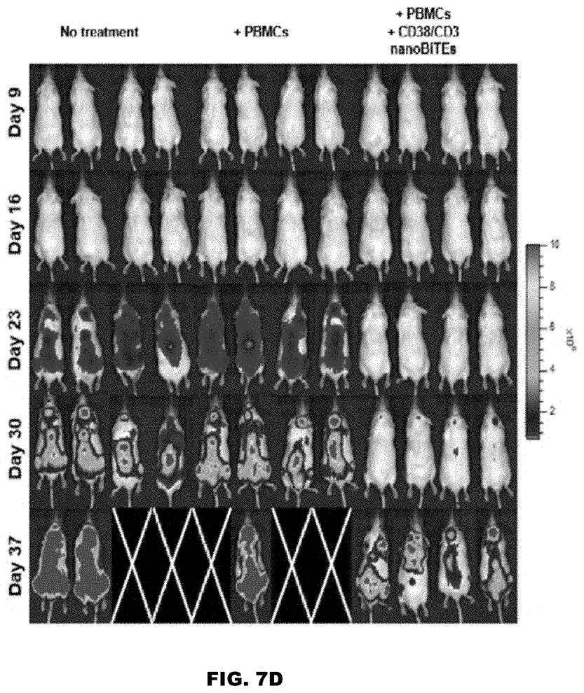

To demonstrate the therapeutic efficacy of CD38/CD3 nanoBiTEs, a study with twelve 50-56 day-old NOD.Cg-Prkdc scid Il2rgtm1WjI/SzJ (NSG) mice (Charles River) was conducted. Two million MM.1S (tagged with luciferase) and five million of PBMCs mixed with CD38/CD3 nanoBiTEs (1 mg/mL) were injected at Days 0 and 3, respectively (FIG. 7A). The mice were injected intravenously and luminescence was assessed using bioluminescence imaging (BLI); Injections and BLI were done weekly. FIG. 7C shows the percent survival of NSG mice during this 40-day study. All the mice were alive after the 37th day for the group with PBMCs and CD38/CD3 nanoBiTEs, whereas the majority of the untreated and PBMC-injected only groups died. Images and the line plots for the images are shown in FIG. 7B and FIG. 7D, respectively. In contrast to untreated or mice injected with only PBMCs, mice infused with PBMCs and nanoBiTEs had a significantly lower photon flux at the 37th day (FIG. 7B).

(vi) NanoBiTE Technology can be Applied to Different Cancers Including Myeloma, Lymphoma, Leukemia, and Solid Tumors Such as Lung, Cervix, Colon, Breast and Prostate.

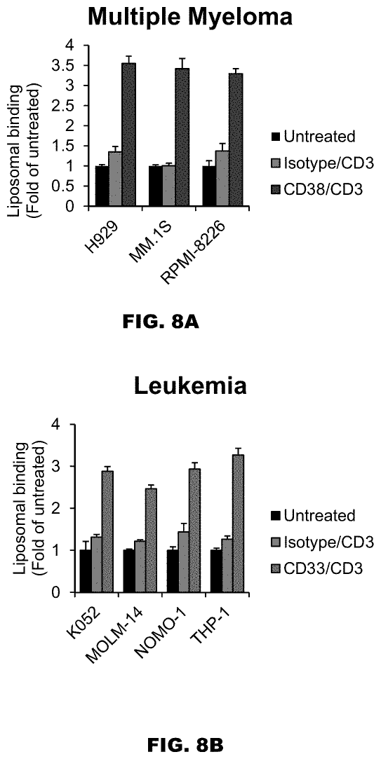

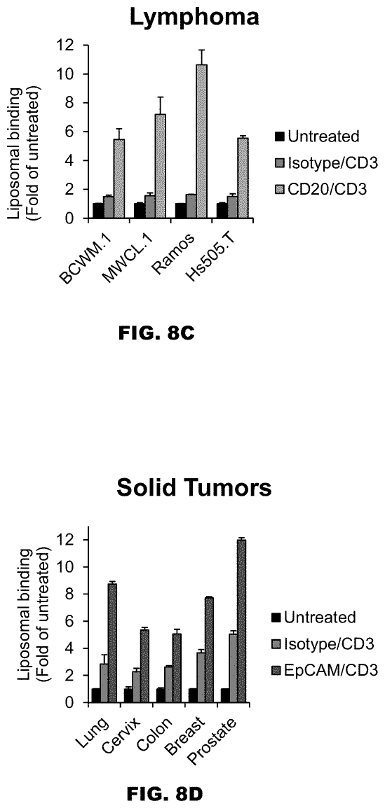

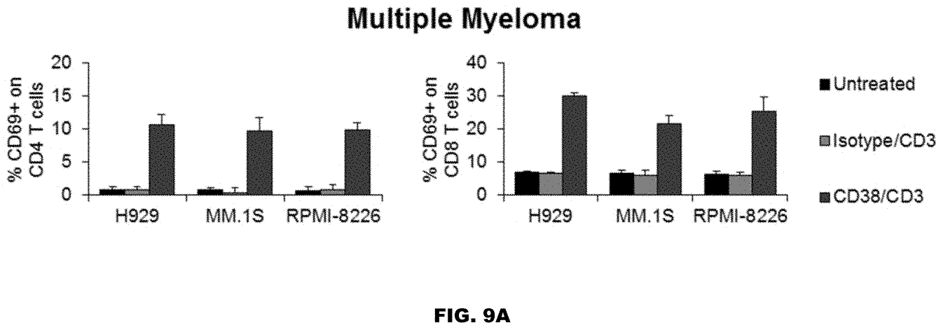

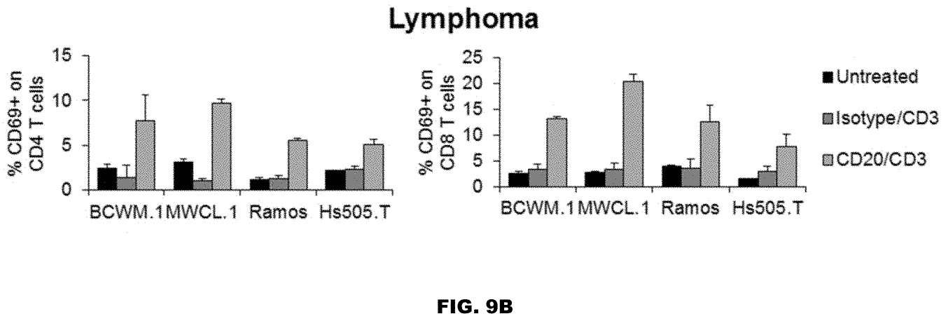

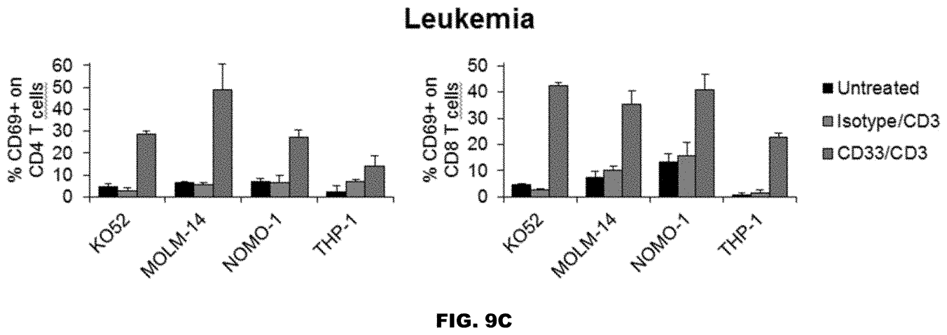

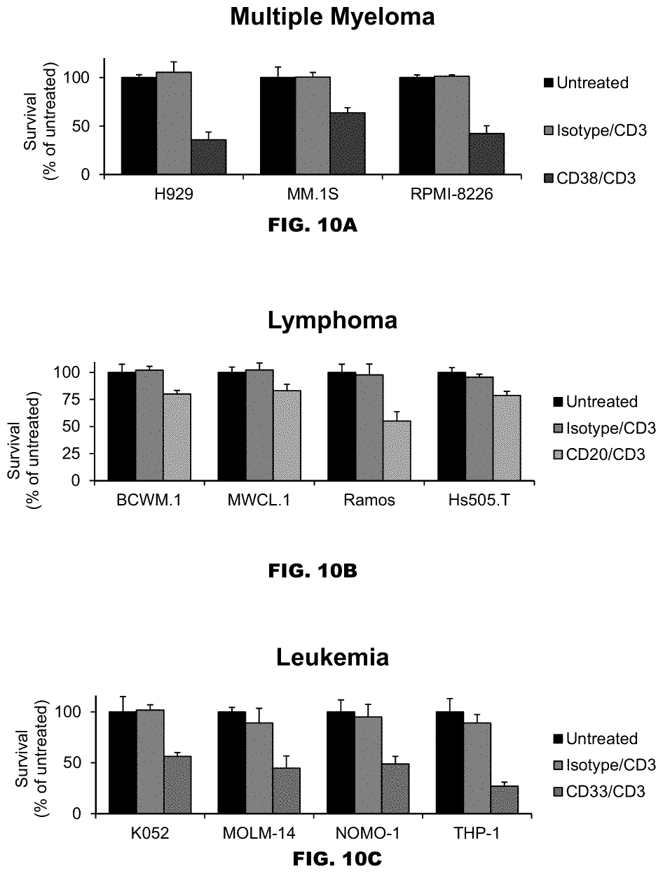

FIG. 8A-8D, FIG. 9A-9C, and FIG. 10A-10C show that various nanoBiTE compositions can be designed to target and redirect T cells to different types of cancers including Myeloma, Lymphoma, leukemia, and solid tumors such as lung, cervix, colon, breast and prostate. This data further highlights the flexibility of the present disclosure to target various types of cancer cells.

(vii) Targeting Multiple Cancer Antigens at the Same Time: In Vitro and In Vivo

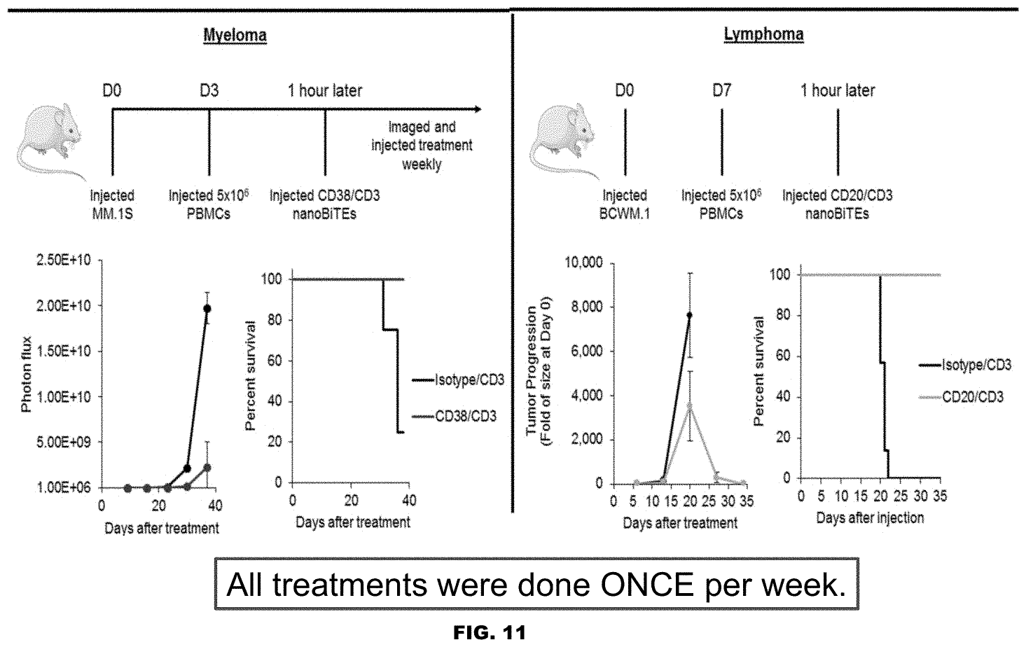

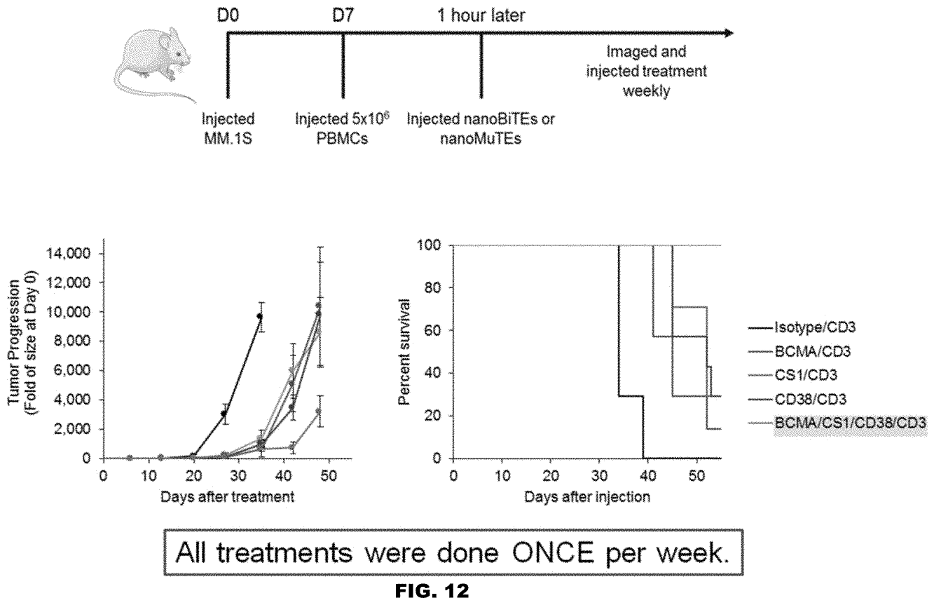

FIG. 11 and FIG. 12 show that targeting multiple antigens at the same time (Nano MuTE's) produces a better response than targeting cancer one antigen at a time. This solves a problem in the field where after treatment with BiTEs and/or CAR-T cells (traditionally targeting one cancer antigen), there is development of “antigen-escape” in which cancer cells with no or low expression of one antigen escape the therapy. In this approach of targeting multiple antigens at the same time, using NanoMuTE's, achieves better efficacy in inhibiting tumor progression and prolonging the survival of animals.

(viii) Pharmacokinetics of the NanoBiTEs

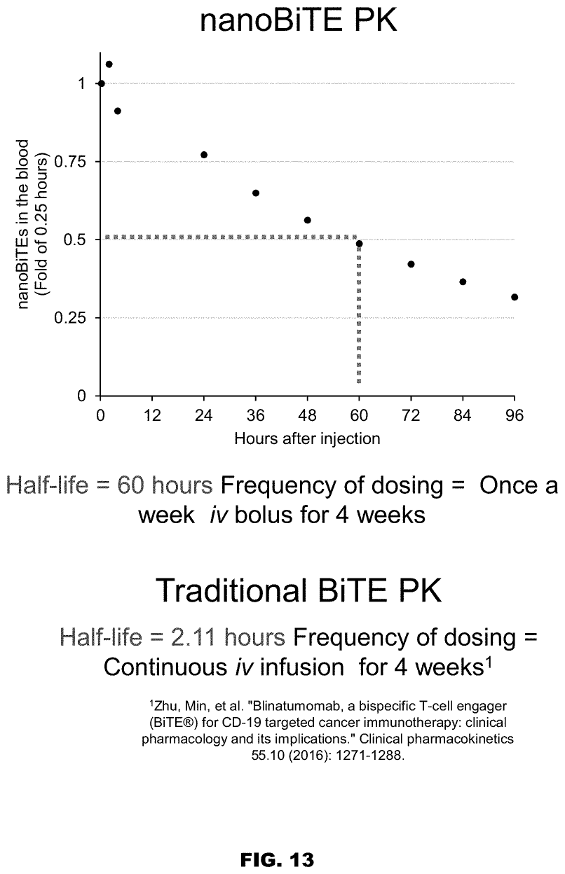

FIG. 13 shows the pharmacokinetics of the NanoBiTEs, that now depends on the liposome part of the NanoBiTE, show long half-life (60 hours) compared with the very short half-life of the regular BiTEs published in the literature (about 2 hours). This solves a problem in the field in which regular BiTEs need to be dosed frequently using a continuous iv infusion for several weeks; while the NanoBiTEs can be conveniently administered using a single IV bolus injection once a week. Such a dose regimen was demonstrated in our in vivo experiments.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A nanoparticle composition comprising: (a) at least one antigen binding moiety; and (b) a T cell moiety; wherein the antigen binding moiety and the T cell moiety are conjugated to the surface of a nanoparticle and wherein the antigen binding moiety specifically binds to an antigen on a cancer cell and the T cell moiety specifically binds to a T cells.

2. The composition of claim 1, comprising 2, 3, 4, or more antigen binding moieties.

3. The composition of claim 2, wherein a first antigen binding moiety binds to a first antigen on a target cell and a second antigen binding moiety binds to a second antigen on a target cell.

4. The composition of claim 3, further comprising a third antigen binding moiety which binds to a third antigen on a target cell.

5. The composition of claim 1, wherein the antigen binding moiety is an antibody, the T cell moiety is an antibody, or both the antigen binding moiety and the T cell moiety are antibodies.

6. The composition of claim 4, wherein the first antigen biding moiety is an antibody which specifically binds CD38, the second antigen binding moiety is an antibody which specifically binds B Cell Maturation Antigen (BCMA), and the third antigen binding moiety is an antibody which specifically bind to CS1.

7. The composition of claim 1, wherein the T cell moiety specifically binds CD3.

8. The composition of claim 1, wherein the nanoparticle is a liposomal nanoparticle.

9. The composition of claim 8, wherein the liposomal nanoparticle is avidin-conjugated.

10. The composition of claim 9, wherein the antigen binding moiety and T cell moiety are biotinylated.

11. A pharmaceutical composition, comprising: the nanoparticle composition of claim 1 and a pharmaceutically acceptable carrier.

12. A method of killing a cancer cell in a subject, the method comprising: administering a therapeutically effective amount of a nanoparticle composition comprising, (a) at least one antigen binding moiety, and (b) a T cell moiety; wherein the antigen binding moiety and the T cell moiety are conjugated to the surface of a nanoparticle, the antigen binding moiety specifically binds to an antigen on a cancer cell and the T cell moiety specifically binds to a T cell and, wherein the composition redirects the T cell to the cancer cell and increases T cell mediated lysis of the cancer cell relative to a subject who has not been administered the nanoparticle composition.

13. The method of claim 12, wherein the nanoparticle composition comprises 2, 3, 4, or more antigen binding moieties.

14. The method of claim 13, wherein a first antigen binding moiety binds to a first antigen on a cancer cell and a second antigen binding moiety binds to a second antigen on a cancer cell.

15. The method of claim 14, wherein the first cancer cell has a distinct molecular signature relative to the second cancer cell.

16. The method of claim 13, further comprising a third antigen binding moiety which binds to a third antigen on a target cell.

17. The method of claim 12, wherein the antigen binding moiety is an antibody, the T cell moiety is an antibody, or both the antigen binding moiety and the T cell moieties are antibodies.

18. The method of claim 16, wherein the first antigen biding moiety is an antibody which specifically binds CD38, the second antigen binding moiety is an antibody which specifically binds B Cell Maturation Antigen (BCMA), and the third antigen binding moiety is an antibody which specifically bind to CS1.

19. The method of claim 12, wherein the T cell moiety specifically binds CD3.

20. The method of claim 12, wherein the subject has multiple myeloma, lymphoma, leukemia, lung cancer, cervical cancer, breast cancer, or prostate cancer.
